(19) United States
(12) Patent Application Publication
(10) Pub. No.: US 2012/0128613 A1
(43) Pub. Date: May 24, 2012

(54) COMPOSITIONS COMPRISING A SKIN-LIGHTENING RESORCINOL AND A SKIN DARKENING AGENT

(76) Inventors: Steven Cochran, Princeton, NJ (US); Menas G. Kizoulis, Bound Brook, NJ (US); Michael Southall, Lawrenceville, NJ (US); Michael Tyerech, Ringoes, NJ (US)

(21) Appl. No.: 13/362,367

(22) Filed: Jan. 31, 2012

Related U.S. Application Data
(63) Continuation of application No. 12/572,650, filed on Oct. 2, 2009.

(51) Int. Cl. A61K 8/41; A61Q 19/02

(52) U.S. Cl. 424/62

(57) ABSTRACT

The present invention relates to a composition comprising a skin-lightening resorcinol and a skin darkening agent. The composition may be topically applied to the skin, such as to treat signs of aging.

### FIELD OF THE INVENTION

A composition comprising a skin-lightening resorcinol and a skin darkening agent is provided. The composition is useful, for example, for topical application to the skin.

### BACKGROUND OF THE INVENTION

A vast array of cosmetic agents are known to aid in modifying the appearance of one's skin. The effect can be direct and immediate. For example, the agent may be a simple reflectant or pigment (such as those in “make-up” or “foundation”) that is applied to the surface of the skin, thereby causing skin to appear more pleasant and even. Another example of agents capable of providing virtually immediate changes in appearance are “so-called” tensioning polymers. Tensioning polymers act by pulling the skin tight as they dry, thereby reducing the appearance of wrinkles, a common sign of aging.

Changes in appearance can also be less direct and develop over time. For example, sunscreens, by screening out ultraviolet light, can indirectly assist in changing the appearance of the skin by reducing near term redness, or reducing the development of darkening or tanning that would otherwise develop via interaction of the skin with ultraviolet light.

It is also known to apply agents that affect the appearance of the skin by penetrating the skin to some depth and interacting biologically with the skin and other tissues. For example, retinoids can affect skin appearance by interacting biologically with the body to enhance the formation of collagen and elastin, also potentially reducing the appearance of wrinkles in the skin.

It is also known that certain skin darkening agents such as dihydroxyacetone chemically react with skin proteins, causing the skin to take on a darker tone. Other skin darkening agents, essentially synthetic versions of MSH (melanin stimulating hormone) can darken the skin by, for example, stimulating the production of melanin, a dark pigment found in skin melanocytes. Working in the contrary manner, certain skin lightening or skin whitening agents such as hydroquinone and extracts of soy can affect skin appearance by reducing the production of tyrosinase, an enzyme which is needed for the skin to produce melanin.

One particular class of compounds known for skin-lightening or skin-whitening effects are alkyresorcinols. For example, it has been reported that purified hexylresorcinol is a suitable skin lightening or skin whitening agent (see US Patent Application Publication No. 2008/0305059 to Chaudhuri et al.).

Applicants have now unexpectedly discovered that topical application of skin-lightening resorcinols can actually promote the formation of such building blocks of the skin as elastin and collagen. Applicants have also found that skin-lightening resorcinols can actually quite unexpectedly be combined with skin darkening agents, thereby offering the opportunity to enhance the formation of elastin and collagen without necessarily causing what may be an undesirable lightening or whitening of the skin.

### SUMMARY OF THE INVENTION

The invention provides a composition comprising a skin-lightening resorcinol and a skin darkening agent.

According to another aspect, the invention provides a method of treating a sign of skin aging comprising topically applying to skin in need of such treatment a composition comprising a skin-lightening resorcinol and a skin darkening agent.

Other features and advantages of the present invention will be apparent from the detailed description of the invention and from the claims.

### DETAILED DESCRIPTION OF THE INVENTION

It is believed that one skilled in the art can, based upon the description herein, utilize the present invention to its fullest extent. The following specific embodiments are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference. Unless otherwise indicated, a percentage or concentration refers to a percentage or concentration by weight (i.e., % (W/W). Unless stated otherwise, all ranges are inclusive of the endpoints, e.g., “from 4 to 9” includes the endpoints 4 and 9.

In certain embodiments, compositions of the present invention are suitable for darkening the skin. As used herein, “darkening” is darkening the appearance of human skin or hair, including, but not limited to, darkening human skin to either achieve a “sun tan” effect or to cover light areas of the skin (e.g., as a result of a scar or a disease or a therapy) or darkening natural hair color or restoring discolored hair due to aging (e.g., gray or white hair) or external aggressions (e.g., excess exposure to sun or chlorine). While throughout the specification reference is made to darkening the skin, it is believed that other keratinous bodily materials such as hair may also be darkened.

Products described herein may optionally be in finished packaged form. In one embodiment, the package is a container such as a plastic, metal or glass tube or jar containing the composition. The product may further contain additional packaging such as a plastic or cardboard box for storing such container. In one embodiment, the product comprises a composition of the invention and contains instructions directing the user to apply the composition to the skin or hair to darken the skin (e.g., to tan the skin), even skin tone (e.g., to darken light areas of the skin or to treat or prevent mottled hyperpigmentation), darken the hair (e.g., to darken light brown, blonde, gray or white hairs), or treat one or more signs of skin aging as set forth below. Such instructions may be printed on the container, label insert, or on any additional packaging.

As used herein, “topically applying” means directly laying on or spreading on outer skin, the scalp, or hair, e.g., by use of the hands or an applicator such as a wipe, roller, or spray.

As used herein, “cosmetically acceptable” means that the ingredients the term describes are suitable for use in contact with tissues (e.g., the skin or hair) without undue toxicity, incompatibility, instability, irritation, allergic response, or the like.

As used herein, a “skin darkening effective amount” means an amount sufficient to induce a darkening of human skin or hair. This amount will vary with the area being treated, the age and skin or hair type of the end user, the duration and nature of the treatment, the specific composition employed, the carrier utilized, and like factors.

In certain embodiments, compositions of the present invention are suitable for treating signs of skin aging. As used herein, “signs of skin aging” includes the presence of lines and wrinkles, loss of elasticity, uneven skin, and blotchiness. In a particularly preferred embodiment, the sign of aging is the presence of lines and wrinkles.

As used herein, “treating signs of skin aging” refers to mitigating, reducing, preventing, improving, or eliminating the presence or signs of skin aging described above.

As used herein, “wrinkle” includes fine lines, fine wrinkles, or coarse wrinkles. Examples of wrinkles include, but are not limited to, fine lines around the eyes (e.g., “crow's feet”), forehead and cheek wrinkles, frown-lines, and laugh-lines around the mouth.

As used herein, “loss of elasticity” includes loss of elasticity or structural integrity of the skin or tissue, including but not limited to sagging, lax and loose tissue. The loss of elasticity or tissue structure integrity may be a result of a number of factors, including but not limited to disease, aging, hormonal changes, mechanical trauma, environmental damage, or the result of an application of products, such as a cosmetics or pharmaceuticals, to the tissue.

As used herein, “uneven skin” means a condition of the skin associated with diffuse or mottled pigmentation, which may be classified as hyperpigmentation, such as post-inflammatory hyperpigmentation.

As used herein, “blotchiness” means a condition of the skin associated with redness or erythema.

As used herein, “cosmetic” refers to a beautifying substance or preparation which preserves, restores, bestows, simulates, or enhances the appearance of bodily beauty or appears to enhance the beauty or youthfulness, specifically as it relates to the appearance of tissue or skin.

As used herein, “cosmetically effective amount” means an amount of a physiologically active compound or composition sufficient for treating one or more signs of skin aging, but low enough to avoid serious side effects. The cosmetically effective amount of the compound or composition will vary with the particular condition being treated, the age and physical condition of the end user, the severity of the condition being treated/prevented, the duration of the treatment, the nature of other treatments, the specific compound or product/composition employed, the particular cosmetically-acceptable carrier utilized, and like factors.

It is believed that one skilled in the art can, based upon the description herein, utilize the present invention to its fullest extent. The following specific embodiments are to be construed as merely illustrative, and not limitative of the remainder of the disclosure in any way whatsoever.

### Skin-Lightening Resorcinol

Resorcinol is a dihydroxy phenol compound (i.e., 1,3-dihydroxybenzene), having the following structure:

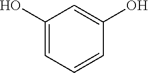

The skin-lightening resorcinols used herein are “substituted resorcinols.” As used herein, “substituted resorcinol” means resorcinol comprising at least one substituent in the 2, 4, 5, or 6 position. Thus, the skin-lightening resorcinol may have as few as one and as many as four substituents. Positions 1 and 3 of the skin-lightening resorcinol preferably comprise an —OH group as shown above; however one or both —OH groups may be replaced by an —OR group in which R is a C₁-C₁₂ alkyl or acyl group.

Skin-lightening resorcinols are disclosed for example in U.S. Pat. No. 4,959,393 (Torihara, et al.), U.S. Pat. No. 6,863,897 (Love et al.) and US Published Patent Application No. 2008/0305059 (Chaudhari et al.), the disclosures of which are incorporated herein by reference.

In certain preferred embodiments, at least one substituent on the skin-lightening resorcinol comprises 2 to 18 carbon atoms, preferably 2 to 12 carbon atoms, more preferably 5 to 10 carbon atoms, even more preferably 5 to 9 carbon atoms, most preferably 5 to 8 carbon atoms. In certain other embodiments, at least one substituent comprises a (linear or branched) alkyl group, such as one having the number of carbon atoms described above. Preferably, at least one substituent comprises an alkyl group that is unsaturated and linear.

In certain embodiments, the 4 position of the skin-lightening resorcinol is substituted, and, in certain embodiments, only the 4 position is substituted. In another embodiment, the 4 position is substituted with an alkyl group. In certain preferred embodiments, the skin-lightening resorcinol comprises a single substituent at the 4 position that comprises an alkyl group. In certain other preferred embodiments, the skin-lightening resorcinol comprises a single substituent at the 4 position that consists of an alkyl group directly bonded to the benzene ring.

Particularly suitable skin-lightening resorcinols include 4-hexyl resorcinol and 4-octylresorcinol, particularly 4-hexyl resorcinol. The structures of 4-hexylresorcinol and 4-octylresorcinol are shown below:

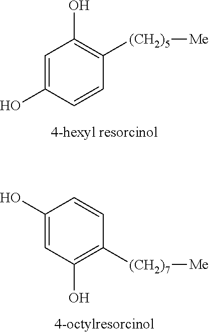

4-Hexyl resorcinol is commercially available as “SYNOVEA HR” from Sytheon of Lincoln Park, N.J. 4-Octylresorcinol is commercially available from City Chemical LLC of West Haven, Conn.

The skin-lightening resorcinol is present in the composition in a safe and effective amount, such as from about 0.01% to about 10%, preferably from about 0.1% to about 5%, more preferably from about 0.2% to about 2%, even more preferably from about 0.5% to about 1.5%, by weight of the composition.

### Skin Darkening Agent

The compositions of the invention comprise a skin darkening agent. As used herein, “skin darkening agent” means an organic moiety or molecule capable of darkening the skin when topically applied thereto, and in particular, an organic moiety or molecule that is capable of darkening the skin without the skin being exposed to UV-radiation.

In a preferred embodiment, the skin darkening agent is capable of providing sufficient darkening to the skin, so as to provide a negative change in darkness relative to a control, −% dL, that is at least about 5%, preferably at least about 15%, more preferably at least about 30%, when tested according to the IN-VIVO SKIN DARKENING TEST evaluated at 3, 6 and/or 24 hours. The IN-VIVO SKIN DARKENING TEST is performed as follows. Two human subjects having Fitzpatrick Skin Types I-III, age ranging from 21-50 are used. For each subject, a location for each of the compositions to be tested is determined and initial chromometer X readings are taken there using a chromometer such as a CR-400 chromometer available from Konica Minolta Sensing of Ramsey, N.J. These readings are averaged to arrive at a baseline reading for each subject. A test sample is prepared by mixing a skin darkening agent in a suitable solvent in order to evenly dissolve or suspend the skin darkening agent. The solvent is preferably water, propylene glycol, or a mixture thereof, but if the skin darkening agent is difficult to dissolve or evenly distribute, up to 50% ethanol may be used, such as 50% ethanol/50% water. A control (no skin darkening agent, only solvent) and the test composition are each separately applied to a distinct area of the forearm for each subject at a coverage of 18 mg over 9 cm² of skin area and are rubbed in. The compositions are allowed to dry and after three time periods, 3 hours, 6 hours, and 24 hours, additional chromometer readings are taken. The change in lightness, dL, is calculated by subtracting the average reading after treatment to average reading of baseline. A change in lightness will typically be negative (indicating a darkening effect) for a skin darkening agent.

The change in lightness for a test sample is normalized to that of the control. This normalized change in lightness is calculated as:

% dL=100*(dL_{test sample}−dL_{control})/dL_{control}

Typically, the change in lightness will be negative for application of a skin darkening agent. As such, in order to avoid using negative numbers, one may refer to the

normalized change in darkness=−% dL

Suitable classes of skin darkening agents include melanin enhancement agents, self-tanning agents, and organic pigments. Preferred skin darkening agents are melanin enhancement agents.

In one embodiment, the skin darkening agent is a melanin enhancement agent, e.g., an agent suitable for enhancing either melanin synthesis (e.g., by enhancing tyrosinase) or for enhancing the transport of melanin to keratinocytes.

Suitable melanin enhancement agents may be selected based on the IN-VITRO SKIN-DARKENING TEST described in U.S. Patent Application Publication No. US2008/0249029 entitled “Methods For Treating Skin Pigmentation,” incorporated herein by reference in its entirety. In the IN-VITRO SKIN-DARKENING TEST, an in vitro epidermal equivalent system is used. The epidermal equivalent system contains melanocytes. One epidermal equivalent system useful in performing this study is the MelanoDerm system, available commercially from MatTek (Ashland, Mass.). This system contains human normal melanocytes, together with normal, human-derived epidermal keratinocytes, which have been cultured to form a multi layered, highly differentiated model of the human epidermis. To demonstrate an increase in pigmentation, equivalents are treated with test compounds, in the absence of ultraviolet light or solar exposure, for three days and samples are harvested on the fourth day after beginning of treatment. The harvested equivalents are then stained with DOPA (a substrate for tyrosinase) and H&E (a standard histological stain) or with Fontana-Mason (F&M) staining, another stain known to those of skill in the art. At least three sections per equivalent, three equivalents per experiment are processed. F&M staining is a silver staining technique that clearly and cleanly marks melanins which have high silver nitrate reducing activity. F&M stained sections can be used for image analysis using Optomax Image Analysis Systems, from Optomax Inc. (Hollis, N.H.) used on a Gateway 2000 PS-100 computer (Media Cybernetics, Silver Springs, Md.) for capturing images. Image Pro Plus version 4.0 can be used for image analysis. Parameters that are measured include: (1) level of pigmentation within individual melanocytes and (2) number of pigmented melanocytes per field, for the Optomax system, or (1) the surface area of silver deposits within melanocytes and (2) the number of pigmented melanocytes for the Image Pro system. Using the Optomax system, surface area of silver deposits within individual melanocytes can be measured in 60 melanocytes, using multiple sections from triplicate equivalents per treatment. The number of melanocytes per field can be calculated in these sections. A “pigmentation factor” is defined as the average surface area of silver deposits within an individual melanocyte, multiplied by the number of pigmented melanocytes per field. A value of one is assigned to untreated controls, and values of treatment groups are normalized to their relevant controls. Suitable melanin enhancement agents have a pigmentation factor of at least 1.2, preferably at least about 1.5, and most preferably at least about 2.

Examples of such melanin enhancement agents include peptides and certain natural extracts, such as extracts of rhubarb, for example those disclosed in U.S. Pat. No. 6,926,886, and extracts of Onosis spinosa (such as “Bugrane-P” a commercially available extract from Silab of Cedex, France).

Examples of suitable skin-darkening peptides are described, e.g., in U.S. Pat. Nos. 7,214,655; 6,797,697; and 7,025,951. Suitable examples of skin-darkening peptides include those having the formula:

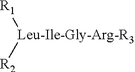

wherein each R₁ and R₂ are independently H, C₁₋₁₂ alkyl, C₇₋₁₀-phenylalkyl, or C(═O)E₁, in which E₁ is C₁₋₂₀ alkyl, C₃₋₂₀ alkenyl, C₃₋₂₀ alkynyl, phenyl, 3,4-dihydroxyphenylalkyl, naphthyl, or C₇₋₁₀-phenylalkyl; provided that when either R₁ or R₂ is C(═O)E₁, the other must be H; and R₃ is OH, NH₂, C₁₋₁₂alkoxy, C₇₋₁₀ phenylalkoxy, C₁₁₋₂₀naphthylalkoxy, C₁₋₂alkylamino, C₇₋₁₀-phenylalkylamino, or C₁₁₋₂₀ naphthylalkylamino, and cosmetically acceptable salts thereof.

Another suitable peptide is Palmitoyl-Val-Gly-Val-Ala-Pro-Gly-NH₂.

Further melanin enhancement agents particularly suitable for use in the present invention are amine compounds of formula I or formula II, shown below:

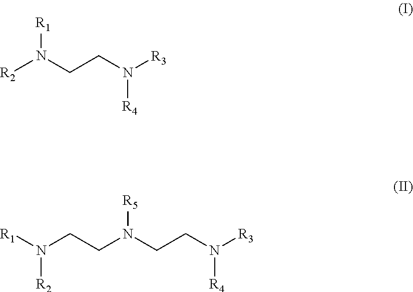

wherein R₁, R₂, R₃, R₄, and R₅ are independently selected from the group consisting of hydrogen, C₁-C₆ alkyl, and C₁-C₆ hydroxyalkyl; or a cosmetically-acceptable salt thereof. Examples of preferred amine compounds of formula I include, but are not limited to, N,N,N′,N′-tetrakis(2-hydroxypropyl)ethylenediamine (THPED), N,N,N′,N′-tetrakis (2-hydroxyethyl)ethylene diamine (THEED), and N,N,N′,N′-tetramethylethylene diamine (TEMED), the structures of which are set forth below, enantiomers thereof, diastereoisomers thereof, or cosmetically-acceptable salts thereof.

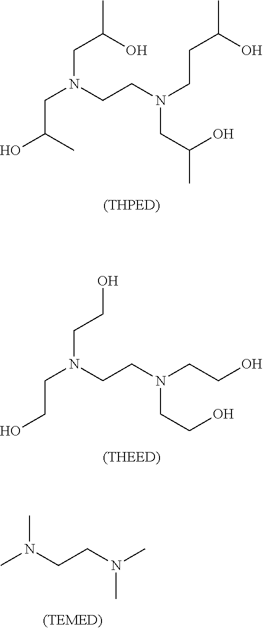

The synthesis of N,N,N′,N′-tetrakis (2-hydroxypropyl)ethylenediamine from the reaction of ethylenediamine with propylene oxide is described in U.S. Pat. No. 2,697,118.

In one embodiment, the skin darkening agent is a self-tanning agent. By self-tanning agent, it is meant a chemical agent capable of producing or inducing the artificial tanning process of the skin by forming brown pigments in the skin via a reaction between a ketose or other suitable functional groups present in the self-tanning agent and amines/amino acids present in the skin, e.g., through the Maillard reaction reported in Bobin, et al., J. Soc. Cosmet. Chem., 35:265-72 (1984) or the Michael's reaction.

In particular, the self-tanning agent may be an α-hydroxy ketone or aldehyde of the formula:

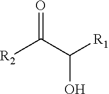

wherein R₁ is H, CH₂OH, CHOHCH₂OH, CH(OH)CH(═O), CH(NH₂)CH(═O), CH(OCH₃)CH(═O), or CH(NH-Phenyl)CH(═O); and R₂ is H or CH₂OH. Examples of such compounds include 1,3-dihydroxyacetone (i.e., dihydroxyacetone, DHA) and 1,3,4-trihydroxy-2-butanone (i.e., erythulose). In a preferred embodiment, the self-tanning agent is 1,3-dihydroxyacetone. In another embodiment, the self-tanning agent is lawsone.

Furthermore, to enhance the effect of the self-tanning agent, compositions of the present invention may include one or more darkening enhancement ingredients to assist the self-tanning agent in developing a darkening effect either more rapidly in time or more intensely. As such, the composition may include for example, sugar amines such as glucosamine, N-acetyl glucosamine, mannosamine, N-acetyl mannosamine, galactosamine, N-acetyl galactosamine; or various natural extracts. Suitable natural extracts include walnut tree extract (e.g., a mixture of water and Juglans regia seed having a 27% to 33% dry matter content, commercially available from Gattefosse of Saint-Priest, France and sold under the name “Gatuline Age Defense”), mugwort extract (e.g., a mixture of mugwort extract and Ulvaceae green algae extract, commercially available as “Triple A Complex,” from Barnet Products Corporation of Englewood Cliffs, N.J.), and certain pre-germinated soy seed extracts (e.g., “Phylderm Vegetal C,” commercially available from Gattefosse of Saint-Priest, France). The amount of the one or more darkening enhancement ingredients in the compositions of the invention, if included, may range from, on an individual basis or in combination with one another, about 0.01% to about 10%, preferably from about 0.1% to about 5%, more preferably from about 0.5% to about 2% by weight of the composition.

In one embodiment, the skin darkening agent is an organic pigment. Preferred organic pigments have 70% or more (preferably 80% or more, more preferably 90% or more) of their molecular weight collectively derived from hydrogen, carbon, nitrogen, oxygen, or phosphorus atoms. Inorganic pigments are not acceptable as skin darkening agents, since these pigments, despite often providing visible light absorption across selected portions of the visible spectrum, typically sit on top of the skin and enhance the intensity of reflected or scattered light to the viewer. As such, inorganic pigments often do not darken the skin or mask skin lightening effects in an aesthetically desirable way. Examples of such inorganic “surface” pigments that may, in certain embodiments, be desirably excluded from the skin darkening agents include those inorganic pigments, particularly (solid) crystalline inorganic pigments, such as those typically employed in make-up and foundation, e.g., mica, titanium oxide, zinc oxide; other oxides, silicates, or aluminosilciates of moieties such as titanium, zinc, copper, iron, chromium, tungsten and other transition metals; as well as mixtures or blends thereof, including lake pigments and inorganic pigments coated with either organic surface treatments or coated with other inorganic moieties to modify the tactile or suspendibility or dispersibility properties of the pigment. In certain embodiments, compositions of the present invention are essentially free of inorganic pigments (particularly inorganic pigments that have a particle size of greater than about 0.1 microns in the composition). As used herein, “essentially free of inorganic pigments” means containing less than about 1%, preferably less than about 0.5%, more preferably less than about 0.1%, by weight of inorganic pigments.

Suitable organic pigments include polymers such as melanin or synthetic melanin, as well as extracts that contain dark pigments in concentrations suitable to darken the skin. What is meant by an “extract” is a mixture of compounds isolated from a natural source (e.g., a plant, algae, and the like). Examples of suitable natural extracts include but not are not limited to Coleus Forskoli extract, extracts from natural sources containing organic pigments (e.g., brown pigments from plants from the Hedychium genus or Bearberry genus or yellow, orange and red pigments from plants containing carotenoids or canthaxanthins); or carotenoids or canthaxantins.

Examples of suitable synthetic melanin derivatives are disclosed in U.S. Pat. Nos. 5,618,519, 5,384,116, and 5,227,459, as well as in US Patent Application Publication No. 2009/0087395. Examples of commercially available soluble melanin enhancement agents include Melasyn-100™ from San-mar laboratories, Inc. (Elmsford, N.Y.) and MelanZe™ from Zylepsis (Ashford, Kent, United Kingdom).

In another embodiment, the skin darkening agent is a glycerophospholipid having a single fatty acid moiety such as lysophosphatidyl choline.

The skin darkening agent is present in the composition in a safe and effective amount. For example, the amount of skin darkening agent in the compositions of the invention may range from, on an individual basis or in combination with one another, about 0.01% to about 10%, preferably from about 0.1% to about 5%, more preferably from about 0.2% to about 2%, and even more preferably from about 0.5% to about 2%, by weight of the composition.

The ratio of concentration of skin darkening agent to concentration of skin-lightening resorcinol may be varied according to the desired effectiveness of the composition in treating of signs of aging and optionally darkening the skin. For example, the skin darkening agent and skin-lightening resorcinol may be present in a concentration by weight ratio (which is determined by dividing the concentration by weight of the skin darkening agent by the concentration by weight of the skin-lightening resorcinol) of at least about 0.1, preferably at least about 0.25, more preferably from about 0.25 to about 10, such as from about 0.5 to about 5, from about 0.75 to about 5, and even more particularly from about 1.25 to about 5.

### Topical Compositions

The compositions of the present invention are applied topically to human skin or hair. In addition to the skin-lightening resorcinol and the skin darkening agent and the optional darkening enhancement ingredients, the composition may further include a cosmetically acceptable topical carrier that may be from about 50% to about 99.99%, by weight, of the composition (e.g., from about 80% to about 99%, by weight, of the composition). In a preferred embodiment of the invention, the cosmetically acceptable topical carrier includes water.

The compositions may be made into a wide variety of product types that include but are not limited to lotions, creams, gels, sticks, sprays, ointments, cleansing liquid washes and solid bars, shampoos and hair conditioners, hair fixers, pastes, foams, powders, mousses, shaving creams, wipes, patches, hydrogels, film-forming products, facial masks and skin masks, films and make-up such as foundations, and mascaras. These product types may contain several types of cosmetically acceptable topical carriers including, but not limited to solutions, suspensions, emulsions such as microemulsions and nanoemulsions, gels, solids and liposomes. The following are non-limiting examples of such carriers. Other carriers can be formulated by those of ordinary skill in the art.

The compositions useful in the present invention can be formulated as solutions. Solutions typically include an aqueous or organic solvent (e.g., from about 50% to about 99.99% or from about 90% to about 99% of a cosmetically acceptable aqueous or organic solvent). Examples of suitable organic solvents include propylene glycol, polyethylene glycol, polypropylene glycol, glycerol, 1,2,4-butanetriol, sorbitol esters, 1,2,6-hexanetriol, ethanol, and mixtures thereof.

Compositions useful in the subject invention may be formulated as a solution comprising an emollient. Such compositions preferably contain from about 2% to about 50% of an emollient(s). As used herein, “emollients” refer to materials used for the prevention or relief of dryness, such as by preventing the transepidermal loss of water from the skin. Examples of emollients include, but are not limited to, those set forth in the International Cosmetic Ingredient Dictionary and Handbook, eds. Pepe, Wenninger and McEwen, pp. 2930-36 (The Cosmetic, Toiletry, and Fragrance Assoc., Washington, D.C., 9th Edition, 2002) (hereinafter “ICI Handbook”). Examples of particularly suitable emollients include vegetable oils, mineral oils, fatty esters, and the like.

A lotion can be made from such a solution. Lotions typically contain from about 1% to about 20% (e.g., from about 5% to about 10%) of an emollient(s) and from about 50% to about 90% (e.g., from about 60% to about 80%) of water.

Another type of product that may be formulated from a solution is a cream. A cream typically contains from about 5% to about 50% (e.g., from about 10% to about 20%) of an emollient(s) and from about 45% to about 85% (e.g., from about 50% to about 75%) of water.

Although it is preferred that the composition of the present invention includes water, the composition may alternatively be anhydrous or an ointment that includes no water but organic and/or silicone solvents, oils, lipids and waxes. An ointment may contain a simple base of animal or vegetable oils or semi-solid hydrocarbons. An ointment may contain from about 2% to about 10% of an emollient(s) plus from about 0.1% to about 2% of a thickening agent(s). Examples of thickening agents include, but are not limited to, those set forth in the ICI Handbook pp. 2979-84.

The composition may be formulated as an emulsion. If the topical carrier is an emulsion, from about 1% to about 10% (e.g., from about 2% to about 5%) of the topical carrier contains an emulsifier(s). Emulsifiers may be nonionic, anionic or cationic. Examples of emulsifiers include, but are not limited to, those set forth in the ICI Handbook, pp. 2962-71.

Lotions and creams can be formulated as emulsions. Typically such lotions contain from 0.5% to about 5% of an emulsifier(s). Such creams typically contain from about 1% to about 20% (e.g., from about 5% to about 10%) of an emollient(s); from about 20% to about 80% (e.g., from 30% to about 70%) of water; and from about 1% to about 10% (e.g., from about 2% to about 5%) of an emulsifier(s).

Single emulsion skin care preparations, such as lotions and creams, of the oil-in-water type and water-in-oil type are well-known in the cosmetic art and are useful in the subject invention. Multiphase emulsion compositions, such as the water-in-oil-in-water type or the oil-in-water-in-oil type, are also useful in the subject invention. In general, such single or multiphase emulsions contain water, emollients, and emulsifiers as essential ingredients.

The compositions of this invention can also be formulated as a gel (e.g., an aqueous, alcohol, alcohol/water, or oil gel using a suitable gelling agent(s)). Suitable gelling agents for aqueous and/or alcoholic gels include, but are not limited to, natural gums, acrylic acid and acrylate polymers and copolymers, and cellulose derivatives (e.g., hydroxymethyl cellulose and hydroxypropyl cellulose). Suitable gelling agents for oils (such as mineral oil) include, but are not limited to, hydrogenated butylene/ethylene/styrene copolymer and hydrogenated ethylene/propylene/styrene copolymer. Such gels typically contains between about 0.1% and 5%, by weight, of such gelling agents.

The compositions of the present invention can also be formulated into a solid formulation (e.g., a wax-based stick, soap bar composition, powder, or a wipe containing powder).

The compositions useful in the subject invention may contain, in addition to the aforementioned components, a wide variety of additional oil-soluble materials and/or water-soluble materials conventionally used in compositions for use on skin and hair, at their art-established levels.

### Additional Cosmetically Active Agents

In one embodiment, the composition further contains another cosmetically active agent. As used herein, a “cosmetically active agent” is a compound (e.g., a synthetic compound or a compound isolated from a natural source or a natural extract) that has a cosmetic or therapeutic effect on the skin or hair, including, but not limiting to, anti-acne agents, shine control agents, anti-microbial agents, anti-inflammatory agents, anti-mycotic agents, anti-parasite agents, external analgesics, sunscreens, photoprotectors, antioxidants, keratolytic agents, surfactants, moisturizers, nutrients, vitamins, energy enhancers, anti-perspiration agents, astringents, deodorants, firming agents, anti-callous agents, and agents for hair and/or skin conditioning.

In one embodiment, the agent is selected from, but not limited to, the group consisting of hydroxy acids, benzoyl peroxide, D-panthenol, octyl methoxycinnimate, octyl salicylate, homosalate, avobenzone, carotenoids, retinoids such as retinol and retinyl palmitate, ceramides, polyunsaturated fatty acids, essential fatty acids, enzymes, enzyme inhibitors, minerals, hormones such as estrogens, steroids such as hydrocortisone, 2-dimethylaminoethanol, copper salts such as copper chloride, peptides containing copper such as Cu:Gly-His-Lys, coenzyme Q10, peptides, amino acids such as proline, vitamins, lactobionic acid, acetyl-coenzyme A, niacin, riboflavin, thiamin, ribose, electron transporters such as NADH and FADH2, and other botanical extracts such as aloe vera, feverfew, oatmeal and derivatives and mixtures thereof. The cosmetically active agent will typically be present in the composition of the invention in an amount of from about 0.001% to about 20% by weight of the composition, e.g., about 0.005% to about 10% such as about 0.01% to about 5%.

Examples of vitamins include, but are not limited to, vitamin A, vitamin Bs such as vitamin B3, vitamin B5, and vitamin B12, vitamin C, vitamin K, and different forms of vitamin E like alpha, beta, gamma or delta tocopherols or their mixtures, and derivatives thereof.

Examples of hydroxy acids include, but are not limited, to glycolic acid, lactic acid, malic acid, salicylic acid, citric acid, and tartaric acid.

Examples of antioxidants include, but are not limited to, water-soluble antioxidants such as sulfhydryl compounds and their derivatives (e.g., sodium metabisulfite and N-acetyl-cysteine), lipoic acid and dihydrolipoic acid, resveratrol, lactoferrin, and ascorbic acid and ascorbic acid derivatives (e.g., ascorbyl palmitate and ascorbyl polypeptide). Oil-soluble antioxidants suitable for use in the compositions of this invention include, but are not limited to, butylated hydroxytoluene, retinoids (e.g., retinol and retinyl palmitate), tocopherols (e.g., tocopherol acetate), tocotrienols, and ubiquinone. Natural extracts containing antioxidants suitable for use in the compositions of this invention, include, but not limited to, extracts containing flavonoids and isoflavonoids and their derivatives (e.g., genistein and diadzein), extracts containing resveratrol and the like. Examples of such natural extracts include grape seed, green tea, pine bark, and propolis.

### Other Materials

Various other materials may also be present in the composition, as known in the art. These include humectants, pH adjusters, chelating agents (e.g., EDTA), fragrances, dyes, and preservatives (e.g., parabens).

The composition and formulations and products containing such compositions of the present invention may be prepared using methodology that is well known by an artisan of ordinary skill.

### Methods of Use

Compositions of the present invention may be topically applied to mammalian skin, such as skin that is in need of treatment for one or more signs of skin aging as described above. In one embodiment, the compositions are applied to skin in need of treatment for lines and wrinkles and/or loss of elasticity. Compositions of the present may also be applied to skin that is in need of increased pigmentation. The compositions may be applied to the skin in need of such treatment according to a suitable treatment regimen, e.g., every month, every week, every other day, every day, twice a day, or the like.

The following non-limiting example further illustrates the invention.

### Example 1

### Preparation of Inventive Example, Ex. 1

The following skin darkening base, Composition A, was prepared:

| TRADE NAME | INGREDIENT | Wt. % |
|---|---|---|
| Phase A | | |
| | Water | 77.55 |
| Ultrez 10 | Carbomer | 0.75 |
| Versene NA | Disodium EDTA | 0.2 |
| Phase B | | |
| Brij 72 | Steareth-2 | 0.75 |
| Brij 721 | Steareth-21 | 1.50 |
| Finsolv TN | C12-15 Alkyl Benzoate | 2.00 |
| Dow Corning Q7 | Dimethicone | 5.000 |
| 9120 Fluid | | |
| Phase C | | |
| Penonip XB | Preservative | 1.00 |
| Neutrol TE | Tetrahydroxypropyl | 1.25 |
| | ethylenediamine | |
| Phase D | | |
| | Water | 8.5 |
| Neutrol TE | Tetrahydroxypropyl | 1.25 |
| | ethylenediamine | |
| Citric Acid | Citric Acid | 0.25 |
| TOTAL | | 100.00 |

Composition A was blended with the following additional ingredients to produce a composition according to the invention:

| TRADE NAME | INGREDIENT | Wt. % |
|---|---|---|
| Composition A | | 70.0 |
| Transcutol CG | | 5.00 |
| Citric acid, 1% | Citric acid | 0.01 |
| Versene NA | Disodium EDTA | 0.01 |
| Synovea HR | 4-hexylresorcinol | 1.00 |
| Butylene glycol | Butylene glycol | 20.0 |
| Water | Water | 3.98 |
| TOTAL | | 100.0 |

This composition comprised a skin darkening agent, specifically tetrahydroxypropyl ethylenediamine, and a skin-lightening resorcinol, specifically, 4-hexylresorcinol. The concentration by weight ratio of skin darkening agent to skin-lightening resorcinol in the composition was (0.7×2.5%)/1.0%=1.75.

The composition was placed at room temperature as well as at elevated temperature (50° C.) for 7 days. No phase instabilities were observed.

It is understood that while the invention has been described in conjunction with the detailed description thereof, that the foregoing description is intended to illustrate and not limit the scope the invention, which is defined by the scope of the appended claims. Other aspects, advantages, and modifications are within the claims.

1-21. (canceled)

22. A composition comprising:
a skin-lightening resorcinol that is a 4-alkyl resorcinol; and
an amine compound of formula I: 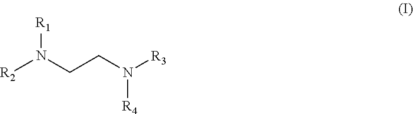
wherein R₁, R₂, R₃, and R₄, are independently selected from the group consisting of hydrogen, C₁-C₆ alkyl, and C₁-C₆ hydroxyalkyl; or a cosmetically-acceptable salt thereof.

23. The composition of claim 22, wherein the skin-lightening resorcinol is 4-hexylresorcinol.

24. The composition of claim 22, wherein the amine is selected from the group consisting of N,N,N′,N′-tetrakis(2-hydroxypropyl)ethylenediamine, N,N,N′,N′-tetrakis (2-hydroxyethyl)ethylene diamine, N,N, N′, N′-tetramethylethylene diamine, enantiomers thereof, diastereoisomers thereof, and cosmetically-acceptable salts thereof.

25. The composition of claim 22, wherein the amine and the skin-lightening resorcinol are present in a concentration by weight ratio of at least about 0.1.

26. The composition of claim 22, wherein the amount of amine is about 0.01% to about 10% by weight of the composition.
